(19) United States
(12) Patent Application Publication
(10) Pub. No.: US 2025/0212963 A1
(43) Pub. Date: Jul. 3, 2025

(54) ATOMIZER AND ELECTRONIC ATOMIZATION APPARATUS

(71) Applicant: Hainan Moore Brothers Technology Co., Ltd., Hainan (CN)

(72) Inventors: Hao YANG, Hainan (CN); Chaoping XU, Hainan (CN); Sanbing REN, Hainan (CN); Guilin LEI, Hainan (CN)

(21) Appl. No.: 19/083,888

(22) Filed: Mar. 19, 2025

Related U.S. Application Data
(63) Continuation of application No. PCT/CN2023/091835, filed on Apr. 28, 2023.

(30) Foreign Application Priority Data
Sep. 20, 2022 (CN) 202211146654.6

(51) Int. Cl. A24F 40/485; A24F 40/10; A24F 40/42; A24F 40/44; A61M 11/04; H05B 3/22

(52) U.S. Cl. CPC A24F 40/485; A24F 40/10; A24F 40/42; A24F 40/44; H05B 3/22; A61M 11/042

(57) ABSTRACT

An atomizer includes: a liquid storage cavity for storing an aerosol-forming medium; and a ventilation channel, a first end of which is communicated with the liquid storage cavity, the ventilation channel having a narrowed portion and/or an expanded portion for preventing the aerosol-forming medium from flowing out of the ventilation channel from a second end of the ventilation channel.

### CROSS-REFERENCE TO PRIOR APPLICATION

This application is a continuation of International Patent Application No. PCT/CN2023/091835, filed on Apr. 28, 2023, which claims priority to Chinese Patent Application No. 202211146654.6, filed on Sep. 20, 2022. The entire disclosure of both applications is hereby incorporated by reference herein.

### FIELD

This application relates to the field of atomizer technologies, and in particular, to an atomizer and an electronic atomization apparatus.

### BACKGROUND

An electronic atomization apparatus generally includes an atomizer and a power supply assembly, and the power supply assembly is electrically connected to the atomizer to provide energy to the atomizer. The atomizer includes a liquid storage cavity. An aerosol-forming medium is stored in the liquid storage cavity. The atomizer is configured to heat and atomize the aerosol-forming medium to generate an aerosol.

To balance the air pressure in the liquid storage cavity, the atomizer is generally provided with a ventilation channel, and the aerosol-forming medium in the liquid storage cavity enters the ventilation channel under an action of capillary force. In the prior art, when the external temperature or pressure changes, the ventilation channel is easily filled with liquid, causing the aerosol-forming medium in the ventilation channel to flow out of the ventilation channel.

### SUMMARY

In an embodiment, the present invention provides an atomizer, comprising: a liquid storage cavity configured to store an aerosol-forming medium; and a ventilation channel, a first end of which is communicated with the liquid storage cavity, the ventilation channel comprising a narrowed portion and/or an expanded portion configured to prevent the aerosol-forming medium from flowing out of the ventilation channel from a second end of the ventilation channel.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic diagram of a structure of an electronic atomization apparatus according to this application;

FIG. 2 a schematic diagram of a structure of a first embodiment of an atomizer of the electronic atomization apparatus provided in FIG. 1;

FIG. 3 is a schematic diagram of a structure of a first embodiment of a mounting base of the atomizer provided in FIG. 2;

FIG. 4 is a schematic diagram of a structure of a first implementation of a ventilation channel of the mounting base provided in FIG. 3;

FIG. 5 is a schematic diagram of a structure of a second implementation of a ventilation channel of the mounting base provided in FIG. 3;

FIG. 6 is a schematic diagram of a structure of a third implementation of a ventilation channel of the mounting base provided in FIG. 3;

FIG. 7 is a schematic diagram of a structure of a fourth implementation of a ventilation channel of the mounting base provided in FIG. 3;

FIG. 8 is a schematic diagram of a structure of a fifth implementation of a ventilation channel of the mounting base provided in FIG. 3;

FIG. 9 is a schematic diagram of a structure of a sixth implementation of a ventilation channel of the mounting base provided in FIG. 3;

FIG. 10 is a schematic diagram of a structure of a seventh implementation of a ventilation channel of the mounting base provided in FIG. 3;

FIG. 11 is a schematic diagram of a structure of an eighth implementation of a ventilation channel of the mounting base provided in FIG. 3;

FIG. 12 is a schematic diagram of a structure of the ventilation channel shown in FIG. 4 during a liquid storage process;

FIG. 13 is a schematic diagram of a structure of the ventilation channel shown in FIG. 4 during a ventilation process;

FIG. 14 is a schematic diagram of a structure of a second embodiment of a mounting base of the atomizer provided in FIG. 2;

FIG. 15 is a schematic diagram of a structure of a first implementation of a ventilation channel of the mounting base provided in FIG. 14;

FIG. 16 is a schematic diagram of a structure of a second implementation of a ventilation channel of the mounting base provided in FIG. 14;

FIG. 17 is a schematic diagram of a structure of a third implementation of a ventilation channel of the mounting base provided in FIG. 14;

FIG. 18 is a schematic diagram of a structure of a fourth implementation of a ventilation channel of the mounting base provided in FIG. 14;

FIG. 19 is a schematic diagram of a structure of the ventilation channel shown in FIG. 17 during a liquid storage process;

FIG. 20 is a schematic diagram of a structure of a third embodiment of a mounting base of the atomizer provided in FIG. 2;

FIG. 21 is a schematic diagram of simulation comparison between a liquid storage process of a third embodiment of an atomizer and a liquid storage process of a comparative example of an atomizer; and

FIG. 22 is a schematic diagram of simulation comparison between a ventilation process of a third embodiment of an atomizer and a ventilation process of a comparative example of an atomizer.

### DETAILED DESCRIPTION

In an embodiment, the present invention provides an atomizer and an electronic atomization apparatus, to resolve a prior art problem that an aerosol-forming medium in a ventilation channel is prone to leakage.

To resolve the foregoing technical problem, a technical solution used in this application is: to provide an atomizer, including:
a liquid storage cavity, configured to store an aerosol-forming medium; and
a ventilation channel, where a first end of the ventilation channel is communicated with the liquid storage cavity; and the ventilation channel includes a narrowed portion and/or an expanded portion to prevent the aerosol-forming medium from flowing out of the ventilation channel from a second end of the ventilation channel.

The ventilation channel includes a main channel section and at least one narrowed section that are communicated with each other, where the narrowed section includes the narrowed portion and/or the expanded portion, or the narrowed section and the main channel section jointly form the narrowed portion and/or the expanded portion; and/or
the ventilation channel includes a main channel section and at least one expanded section that are communicated with each other, where the expanded section includes the narrowed portion and/or the expanded portion, or the narrowed section and the main channel section jointly form the narrowed portion and/or the expanded portion.
the ventilation channel includes the narrowed section, and the equivalent diameter of the narrowed section gradually decreases along a direction from the first end to the second end of the ventilation channel to form a first narrowed portion, and then gradually increases to form a first expanded portion, or the equivalent diameter of the narrowed section remains unchanged along a direction from the first end to the second end of the ventilation channel, and the equivalent diameter of the narrowed section is smaller than the equivalent diameter of the main channel section; or
the ventilation channel includes the expanded section, and the equivalent diameter of the expanded section gradually increases along an extension direction from the first end to the second end of the ventilation channel to form a second expanded portion, and then gradually decreases to form a second narrowed portion, or the equivalent diameter of the expanded section remains unchanged along a direction from the first end to the second end of the ventilation channel, and the equivalent diameter of the expanded section is greater than the equivalent diameter of the main channel section.

The narrowed section further includes a first uniform connection portion communicating the first narrowed portion and the first expanded portion, and the equivalent diameter of the first uniform connection portion remains unchanged along the direction from the first end to the second end of the ventilation channel; or
the ventilation channel includes the expanded section, the expanded section further includes a second uniform connection portion communicating the second narrowed portion and the second expanded portion, and the equivalent diameter of the second uniform connection portion remains unchanged along the direction from the first end to the second end of the ventilation channel.

The narrowed portion or the expanded portion transitions in a curved form along a direction from the first end to the second end of the ventilation channel; or
the narrowed portion or the expanded portion transitions in a linear form along a direction from the first end to the second end of the ventilation channel; or the narrowed portion or the expanded portion transitions in a stepwise form along a direction from the first end to the second end of the ventilation channel.

The ventilation channel includes the main channel section and the at least one narrowed section that are communicated with each other, where the maximum equivalent diameter of the narrowed section is 0.2 mm to 1 mm.

The ventilation channel includes the main channel section and the at least one narrowed section that are communicated with each other, where the minimum equivalent diameter of the narrowed section is smaller than the maximum equivalent diameter; and the ratio of the maximum equivalent diameter to the minimum equivalent diameter of the narrowed section is M, where 1<M<4.

The side wall of the ventilation channel has a lyophilic property.

The material of the side wall of the ventilation channel is a lipophilic material; or the side wall of the ventilation channel is provided with a lipophilic material coating; or
the side wall of the ventilation channel has a lipophilic micro-nano structure. The atomizer includes:
a housing, provided with an air outlet tube and an accommodating cavity; and
a mounting base, disposed in the accommodating cavity, where the mounting base and the housing jointly form the liquid storage cavity; the mounting base is internally provided with an atomization cavity, and the atomization cavity is communicated with the air outlet tube and the external atmosphere;
the ventilation channel is provided in the mounting base; and the second end of the ventilation channel is communicated with the atomization cavity or the external atmosphere.

The ventilation channel is of a linear structure; and
the first end of the ventilation channel is directly communicated with the liquid storage cavity, and the second end is communicated with the atomization cavity.

The atomizer further includes a sealing member, and the outer side surface of the mounting base is provided with a micro groove, and the sealing member covers the micro groove to form the ventilation channel; or
the atomizer further includes a ventilation member, and the ventilation channel is provided in the ventilation member, and the ventilation member is disposed on the mounting base.

The atomizer includes the ventilation member, and the ventilation member is a hollow tube, which is used as the ventilation channel; and a part of the wall of the hollow tube curves inward to form a narrowed section, and the narrowed section includes a first narrowed portion and a first expanded portion; and/or a part of the wall of the hollow tube is expanded outward to form an expanded section, and the expanded section includes a second narrowed portion and a second expanded portion.

The material of the sealing member and/or the material of the mounting base are/is a lipophilic material; or the material of the ventilation member is a lipophilic material.

The atomizer includes only one ventilation channel.

The atomizer further includes an atomization core, and the atomization core is disposed in the mounting base and is fluidly communicated with the liquid storage cavity; and the atomization core includes a porous liquid-guiding substrate and a heating element.

To resolve the foregoing technical problem, another technical solution used in this application is: to provide an electronic atomization apparatus, including:
an atomizer, where the atomizer is the atomizer of any one of the descriptions; and
a power supply assembly, electrically connected to the atomizer, and configured to provide energy to the atomizer.

This application has the following beneficial effects: Different from the prior art, this application discloses an atomizer and an electronic atomization apparatus. The atomizer includes a liquid storage cavity and a ventilation channel. The liquid storage cavity is configured to store an aerosol-forming medium. A first end of the ventilation channel is communicated with the liquid storage cavity. The ventilation channel includes a narrowed portion and/or an expanded portion, to prevent the aerosol-forming medium from flowing out of the ventilation channel from a second end of the ventilation channel. Based on the foregoing configuration, the structure of the narrowed portion and/or the expanded portion of the ventilation channel changes the capillary force direction in the ventilation channel, effectively preventing the aerosol-forming medium in the ventilation channel from flowing out of the ventilation channel from the second end, thereby resolving a prior art problem that the aerosol-forming medium in the ventilation channel is prone to leakage, and improving the performance of the atomizer.

The technical solutions in embodiments of this application are clearly and completely described below with reference to the accompanying drawings in embodiments of this application. Apparently, the described embodiments are merely some rather than all of embodiments of this application. All other embodiments obtained by a person of ordinary skill in the art based on embodiments of this application without creative efforts shall fall within the protection scope of this application.

The terms “first”, “second”, and “third” in embodiments of this application are merely intended for a purpose of description, and shall not be understood as an indication or implication of relative importance or implicit indication of the quantity of indicated technical features. Therefore, features defining “first”, “second”, and “third” may explicitly or implicitly include at least one of the features. In descriptions of this application, “a plurality of” means at least two, such as two and three unless it is specifically defined otherwise. In addition, the terms “include”, “have”, and any variant thereof are intended to cover a non-exclusive inclusion. For example, a process, method, system, product, or device that includes a series of steps or units is not limited to the listed steps or units; and instead, further optionally includes a step or unit that is not listed, or further optionally includes another step or unit that is intrinsic to the process, method, product, or device.

“Embodiment” mentioned in the specification means that particular features, structures, or characteristics described with reference to the embodiment may be included in at least one embodiment of this application. The term appearing at different positions of this specification may not refer to the same embodiment or an independent or alternative embodiment that is mutually exclusive with another embodiment. A person skilled in the art explicitly or implicitly understands that embodiments described in the specification may be combined with other embodiments.

FIG. 1 is a schematic diagram of a structure of an electronic atomization apparatus according to this application.

Refer to FIG. 1. This application provides an electronic atomization apparatus 300, and the electronic atomization apparatus 300 can be used for atomization of an aerosol-forming medium. The electronic atomization apparatus 300 includes an atomizer 100 and a power supply assembly 200 that are electrically connected to each other.

The atomizer 100 is configured to store an aerosol-forming medium and atomize the aerosol-forming medium to form an aerosol that can be inhaled by a user. The atomizer 100 may be specifically used in different fields such as medical care, cosmetology, and recreational vaping. In a specific embodiment, the atomizer 100 may be used in an electronic aerosol atomization apparatus to atomize an aerosol-forming medium and generate an aerosol for inhalation by an inhaler. The following embodiments are described by using an example in which the atomizer is used in the field of recreational vaping.

For a specific structure and functions of the atomizer 100, reference may be made to the specific structure and functions of the atomizer 100 involved in the following embodiments, same or similar technical effects may also be implemented, and details are not described herein again.

The power supply assembly 200 includes a battery and a controller. The battery is configured to supply electric energy for operation of the atomizer 100, to enable the atomizer 100 to heat and atomize the aerosol-forming medium to form an aerosol. The controller is configured to control the operation of the atomizer 100. The power supply assembly 200 also includes other components such as a battery holder and an airflow sensor.

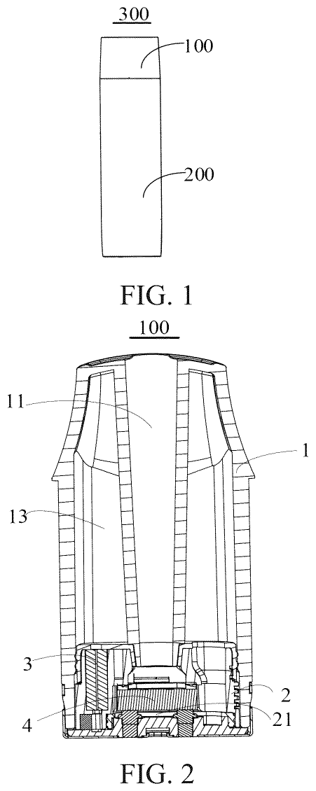

FIG. 2 is a schematic diagram of a structure of a first embodiment of an atomizer of the electronic atomization apparatus provided in FIG. 1.

Refer to FIG. 2. The atomizer 100 of the electronic atomization apparatus 300 provided in this application includes a housing 1, a mounting base 2, and a ventilation channel 3. The housing 1 is provided with an air outlet tube 11 and an accommodating cavity, the mounting base 2 is disposed in the accommodating cavity, the mounting base 2 and the housing 1 jointly form a liquid storage cavity 13, and an aerosol-forming medium is stored in the liquid storage cavity 13. The mounting base 2 is internally provided with an atomization cavity 21 inside, and the atomization cavity 21 is communicated with the air outlet tube 11 and the external atmosphere. The ventilation channel 3 is provided in the mounting base 2, a first end 31 of the ventilation channel 3 is communicated with the liquid storage cavity 13, and a second end 32 of the ventilation channel 3 is communicated with the external atmosphere or the atomization cavity 21.

The side wall of the ventilation channel 3 has a lyophilic property, such as hydrophilicity (hydrophilicity) or lipophilicity (lipophilicity). In this embodiment, the aerosol-forming medium is an oily liquid, and the side wall of the ventilation channel 3 has a lipophilic property. Specifically, the ventilation channel 3 may be made of a lipophilic material, for example, the side wall of the ventilation channel 3 may be made of a PCTG material; or the side wall of the ventilation channel 3 may be coated with a lipophilic material coating, so that the side wall of the ventilation channel 3 has a lipophilic property; or the side wall of the ventilation channel 3 may be configured to have a lipophilic micro-nano structure, so that the side wall of the ventilation channel 3 has a lipophilic property. Because the side wall of the ventilation channel 3 has a lipophilic property, when the aerosol-forming medium enters the ventilation channel 3, a capillary force direction A points from the first end 31 to the second end 32 of the ventilation channel 3.

In this embodiment, the ventilation channel 3 includes a narrowed portion and/or an expanded portion, to prevent the aerosol-forming medium from flowing out of the ventilation channel 3 from the second end 32. The narrowed portion in this application refers to a portion of the ventilation channel 3 where the equivalent diameter changes from large to small, and the expanded portion refers to a portion of the ventilation channel 3 where the equivalent diameter changes from small to large. This change may be abrupt or gradual. The gradual change may follow a linear path, a curved path, or a stepwise path.

It may be understood that, in this embodiment, the narrowed portion and/or the expanded portion are/is provided in the ventilation channel 3 communicating the liquid storage cavity 13 with the external atmosphere/atomization cavity 21, so that the equivalent diameter of the ventilation channel 3 changes at the narrowed portion and/or the expanded portion. As a result, the capillary force direction A in the ventilation channel 3 also changes at the narrowed portion and/or the expanded portion, so as to prevent a problem that the ventilation channel 3 is easily filled with liquid due to the continuous entry of the aerosol-forming medium into the ventilation channel 3 from the liquid storage cavity 13 because the capillary force direction A in the ventilation channel 3 always points from the liquid storage cavity 13 to the external atmosphere/atomization cavity 21, specifically, the capillary force direction A always points from the first end 31 to the second end 32 and remains unchanged, due to the lipophilic property of the side wall of the ventilation channel 3. In this way, a problem of liquid leakage from the atomizer 100 caused by the aerosol-forming medium in the ventilation channel 3 flowing out of the ventilation channel 3 from the second end 32 is prevented, a prior art problem that the aerosol-forming medium in the ventilation channel 3 is prone to leakage is resolved, thereby improving the atomization performance of the atomizer 100.

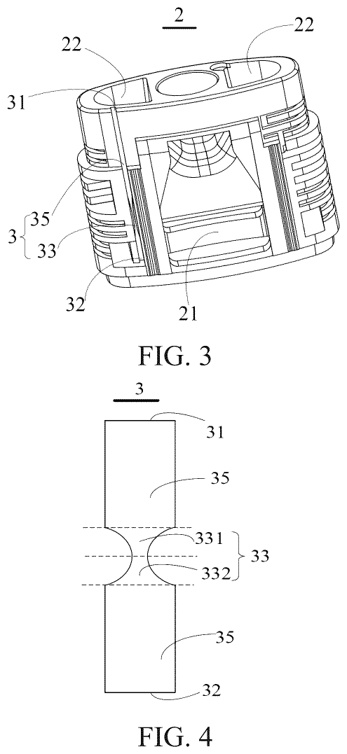

Specifically, the atomizer 100 further includes an atomization core 4. The atomization core 4 is disposed in the mounting base 2. The atomization core 4 and the mounting base 2 jointly form the atomization cavity 21. An atomization process takes place in the atomization cavity 21. The mounting base 2 is provided with a liquid inlet hole 22 (as shown in FIG. 3), the atomization core 4 is fluidly communicated with the liquid storage cavity 13, and the aerosol-forming medium stored in the liquid storage cavity 13 is absorbed by the atomization core 4 via the liquid inlet hole 22 of the mounting base 2. The atomization core 4 includes a porous liquid-guiding substrate and a heating element. The porous liquid-guiding substrate may be made of a porous material such as porous ceramics or porous glass, or may be made of a dense ceramic material with a porous structure formed by creating openings. The heating element may be any metal heating structure, for example, a heating film, a heating wire, or a heating net. The heating element is configured to generate heat when being energized, so as to heat the aerosol-forming medium in the atomization core 4 to atomize and generate the aerosol. The atomization cavity 21 is communicated with the air outlet tube 11, and the aerosol generated by atomization flows to the air outlet tube 11 through the atomization cavity 21 and is finally inhaled by a user.

In this embodiment, the atomizer 100 may include one or more ventilation channels 3. It may be understood that when the atomizer 100 includes only one ventilation channel 3, a prior art problem of aerosol-forming medium leakage in the atomizer 100 that includes two or more ventilation channels 3, in which when the pressure in the plurality of ventilation channels 3 is asymmetric, for example, when the atomizer 100 is tilted, one ventilation channel 3 ventilates while another ventilation channel 3 absorbs liquid, the aerosol-forming medium in the ventilation channel 3 on the liquid absorption side is likely to flow out of the ventilation channel 3, can be resolved. In another implementation, the atomizer 100 may alternatively include a plurality of ventilation channels 3.

The ventilation channel 3 may include a main channel section 35 and a narrowed section 33 and/or an expanded section 34. The narrowed section 33 or the expanded section 34 may include a narrowed portion and/or an expanded portion, or the narrowed section 33 or the expanded section 34 may form a narrowed portion and/or an expanded portion together with the main channel section 35. In one embodiment, the ventilation channel 3 may include only the main channel section 35 and the narrowed section 33 that are communicated with each other. In another embodiment, the ventilation channel 3 may include only the main channel section 35 and the expanded section 34 that are communicated with each other. In another embodiment, the ventilation channel 3 may include a plurality of main channel sections 35, at least one narrowed section 33, and at least one expanded section 34 that are communicated with each other, or may be designed as needed.

In this application, the ventilation channel 3 is provided in the mounting base 2. Specifically, the ventilation channel 3 may be directly formed in the mounting base 2, or the ventilation channel 3 may be configured to be formed in a separate structural member and the separate structural member is mounted on the mounting base 2.

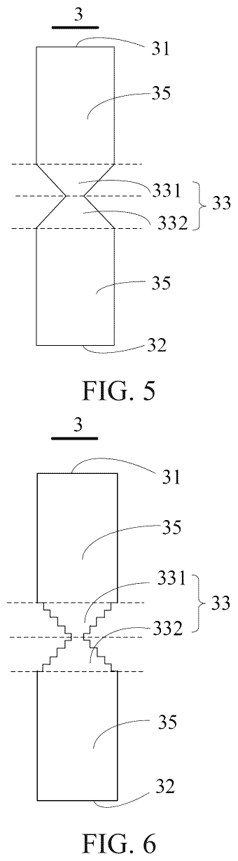

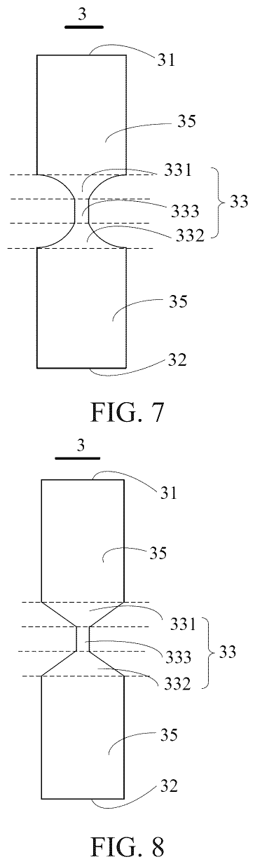

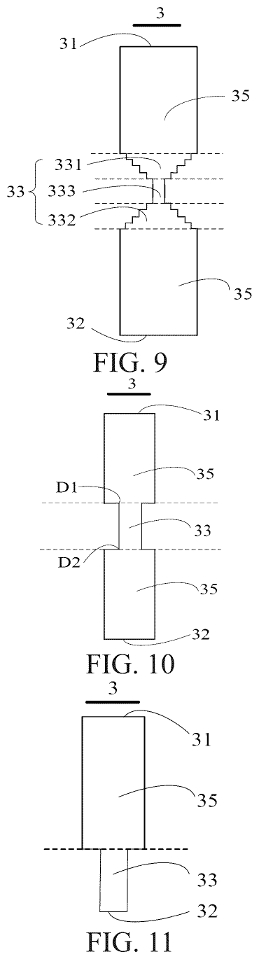

FIG. 3 is a schematic diagram of a structure of a first embodiment of a mounting base of the atomizer provided in FIG. 2. FIG. 4 is a schematic diagram of a structure of a first implementation of a ventilation channel of the mounting base provided in FIG. 3. FIG. 5 is a schematic diagram of a structure of a second implementation of a ventilation channel of the mounting base provided in FIG. 3. FIG. 6 is a schematic diagram of a structure of a third implementation of a ventilation channel of the mounting base provided in FIG. 3. FIG. 7 is a schematic diagram of a structure of a fourth implementation of a ventilation channel of the mounting base provided in FIG. 3. FIG. 8 is a schematic diagram of a structure of a fifth implementation of a ventilation channel of the mounting base provided in FIG. 3. FIG. 9 is a schematic diagram of a structure of a sixth implementation of a ventilation channel of the mounting base provided in FIG. 3. FIG. 10 is a schematic diagram of a structure of a seventh implementation of a ventilation channel of the mounting base provided in FIG. 3. FIG. 11 is a schematic diagram of a structure of an eighth implementation of a ventilation channel of the mounting base provided in FIG. 3.

In this embodiment, the ventilation channel 3 is directly formed in the mounting base 2. Specifically, the atomizer 100 further includes a sealing member, and the sealing member covers the end surface of the mounting base 2 close to the liquid storage cavity 13 and covers the outer side surface of the mounting base 2. The side wall of the sealing member fits against the housing 1 and the mounting base 2 to seal the liquid storage cavity 13, so as to prevent leakage of the aerosol-forming medium in the liquid storage cavity 13. Refer to FIG. 3. The outer side surface of the mounting base 2 is provided with a micro groove, and the sealing member covers the micro groove in the outer side surface of the mounting base 2 to form the ventilation channel 3. Specifically, the sealing member and/or the mounting base 2 may be made of a lipophilic material, so that the formed ventilation channel 3 has a lipophilic property.

As shown in FIG. 3, the ventilation channel 3 is of a linear structure. In this embodiment, the first end 31 of the ventilation channel 3 is directly communicated with the liquid storage cavity 13, and the second end 32 is communicated with the atomization cavity 21. The ventilation channel 3 realizes the liquid storage and ventilation functions under an action of capillary force. Specifically, the external atmosphere enters the second end 32 of the ventilation channel 3 through the atomization cavity 21, and then enters the liquid storage cavity 13 from the first end 31 through the ventilation channel 3 to implement the ventilation process. The aerosol-forming medium in the liquid storage cavity 13 enters the ventilation channel 3 from the first end 31 of the ventilation channel 3 under the action of the capillary force to implement the liquid storage function.

In another implementation, the second end 32 of the ventilation channel 3 may alternatively not be directly communicated with the atomization cavity 21. For example, the second end 32 of the ventilation channel 3 may be directly communicated with the external atmosphere, and the external atmosphere directly enters the ventilation channel 3 through the second end 32 of the ventilation channel 3 to implement the ventilation process.

In this embodiment, the ventilation channel 3 includes only the main channel section 35 and the narrowed section 33. The quantity of the main channel sections 35 and the narrowed sections 33 may be one or more. As shown in FIG. 3, the ventilation channel 3 includes two main channel sections 35 and one narrowed section 33 that are communicated with each other. The narrowed section 33 is located between the two main channel sections 35. The two main channel sections 35 are communicated with the liquid storage cavity 13 and the atomization cavity 21, respectively. In another implementation, the quantity of narrowed sections 33 may be two or more. For example, the ventilation channel 3 may include three main channel sections 35 and two narrowed sections 33. The two narrowed sections 33 are communicated with each other via one main channel section 35. The first end 31 and the second end 32 of the ventilation channel 3 are both main channel sections 35. It may be understood that when the ventilation channel 3 includes a plurality of narrowed sections 33, during the liquid storage process in the ventilation channel 3, the plurality of narrowed sections 33 can prevent the aerosol-forming medium from continuing to flow toward the second end 32. The resistance to the aerosol-forming medium is strong, and the effect is significant, so that the aerosol-forming medium in the ventilation channel 3 can be effectively prevented from flowing out from the second end 32.

In this embodiment, the narrowed portion and/or expanded portion may be directly formed at the narrowed section 33, or may be jointly formed by the narrowed section 33 and the main channel section 35.

Refer to 4 to 6. In one implementation, the narrowed portion and the expanded portion are directly formed at the narrowed section 33. Specifically, the equivalent diameter of the narrowed section 33 first gradually decreases and then gradually increases along a direction from the first end 31 to the second end 32 of the ventilation channel 3. The narrowed section 33 includes a first narrowed portion 331 and a first expanded portion 332. The equivalent diameter of the first narrowed portion 331 gradually decreases along the direction from the first end 31 to the second end 32 of the ventilation channel 3, and the equivalent diameter of the first expanded portion 332 gradually increases along the direction from the first end 31 to the second end 32 of the ventilation channel 3. In other words, the equivalent diameter at a position where the first narrowed portion 331 is connected to the first expanded portion 332 is the smallest. The equivalent diameter of the main channel section 35 remains unchanged along the direction from the first end 31 to the second end 32 of the ventilation channel 3, and the equivalent diameter at a position where the first narrowed portion 331 is connected to the main channel section 35 and the equivalent diameter at a position where the first expanded portion 332 is connected to the main channel section 35 are the largest.

The maximum equivalent diameter of the narrowed section 33 is 0.2 mm to 1 mm, and the minimum equivalent diameter of the narrowed section 33 is smaller than the maximum equivalent diameter. The ratio of the maximum equivalent diameter to the minimum equivalent diameter of the narrowed section 33 is M. In some implementations, 1<M<4. It may be understood that, if the maximum equivalent diameter of the narrowed section 33 is excessively large, the sum of the maximum along-the-way resistance and capillary force is not sufficient to prevent the aerosol-forming medium from flowing out of the ventilation channel 3, and leakage of the aerosol-forming medium in the ventilation channel 3 is likely to occur.

The micro groove in the mounting base 2 may be configured to be of any shape, to form ventilation channels 3 of different shapes. For example, the cross-sectional shape of the ventilation channel 3 may be of any shape such as a circle, a rectangle, a square, or a triangle. The narrowed section 33 of the ventilation channel 3 may transition in any form along the direction from the first end 31 to the second end 32 of the ventilation channel 3.

As shown in FIG. 4, in one implementation, the narrowed section 33 of the ventilation channel 3 transitions in a curved form along the direction from the first end 31 to the second end 32 of the ventilation channel 3, and the side wall of the micro groove, which is provided in the outer side surface of the mounting base 2, at positions corresponding to the first narrowed portion 331 and the first expanded portion 332 is curved. Specifically, the side wall of the micro groove at the positions corresponding to the first narrowed portion 331 and the first expanded portion 332 is arcuate, and the side edges of the first narrowed portion 331 and the first expanded portion 332 on the longitudinal section of the narrowed section 33 are arc lines.

As shown in FIG. 5, in another implementation, the narrowed section 33 of the ventilation channel 3 transitions in a linear form along a direction from the first end 31 to the second end 32 of the ventilation channel 3, the side wall of the micro groove, which is provided on the outer side surface of the mounting base 2, at positions corresponding to the first narrowed portion 331 and the first expanded portion 332 is planar, the side edges of the first narrowed portion 331 and the first expanded portion 332 on the longitudinal section of the narrowed section 33 are linear lines, and the side edges of the narrowed section 33 form a V shape.

As shown in FIG. 6, in another implementation, the narrowed section 33 of the ventilation channel 3 transitions in a stepwise form along a direction from the first end 31 to the second end 32 of the ventilation channel 3, and the side wall of the micro groove, which is provided on the outer side surface of the mounting base 2, at positions corresponding to the first narrowed portion 331 and the first expanded portion 332 is folded, and the side edges of the first narrowed portion 331 and the first expanded portion 332 on the longitudinal section of the narrowed section 33 are stepwise broken lines.

Optionally, the narrowed section 33 may further include a first uniform connection portion 333. The first uniform connection portion 333 is connected between the first narrowed portion 331 and the first expanded portion 332. The equivalent diameter of the first uniform connection portion 333 remains unchanged along the direction from the first end 31 to the second end 32 of the ventilation channel 3. The equivalent diameter of the first uniform connection portion 333 is the minimum equivalent diameter of the first narrowed portion 331 and the first expanded portion 332. The cross-sectional shape of the first uniform connection portion 333 may be any shape, for example, a rectangle, a circle, or a square.

As shown in FIG. 7, in one implementation, the narrowed section 33 includes a first uniform connection portion 333. The first uniform connection portion 333 is connected between the first narrowed portion 331 and the first expanded portion 332. The first narrowed portion 331 and the first expanded portion 332 have same shapes as the first narrowed portion 331 and the first expanded portion 332 in the narrowed section 33 shown in FIG. 4, and both transition in a curved form along the direction from the first end 31 to the second end 32 of the ventilation channel 3.

As shown in FIG. 8, in another implementation, the narrowed section 33 includes a first uniform connection portion 333. The first uniform connection portion 333 is connected between the first narrowed portion 331 and the first expanded portion 332. The shapes of the first narrowed portion 331 and the first expanded portion 332 are the same as the shapes of the first narrowed portion 331 and the first expanded portion 332 in the narrowed section 33 shown in FIG. 5, and both transition a linear line along the direction from the first end 31 to the second end 32 of the ventilation channel 3.

As shown in FIG. 9, in another implementation, the narrowed section 33 includes a first uniform connection portion 333 communicating the first narrowed portion 331 and the first expanded portion 332. The shapes of the first narrowed portion 331 and the first expanded portion 332 are the same as the shapes of the first narrowed portion 331 and the first expanded portion 332 in the narrowed section 33 shown in FIG. 6, and both transition in a stepwise form along the direction from the first end 31 to the second end 32 of the ventilation channel 3.

It may be understood that in another implementation, the narrowed section 33 may alternatively include only the first narrowed portion 331, but not the first expanded portion 332. The first narrowed portion 331 may transition in any form such as a linear line transition form, a curve transition form, or a stepwise transition form along the direction from the first end 31 to the second end 32.

Refer to 10 and 11, in another implementation, the narrowed portion and/or expanded portion are/is jointly formed by the narrowed section 33 and the main channel section 35.

As shown in FIG. 10, in one implementation, the ventilation channel 3 includes two main channel sections 35 and one narrowed section 33. The narrowed section 33 is located between the two main channel sections 35. Sections at the first end 31 and the second end 32 are both the main channel sections 35. In this implementation, the narrowed section 33 does not include the first narrowed portion 331 and the first expanded portion 332, and the equivalent diameter of the narrowed section 33 remains unchanged along a direction from the first end 31 to the second end 32 of the ventilation channel 3, and the equivalent diameter of the narrowed section 33 is smaller than the equivalent diameter of the main channel section 35. Because the equivalent diameter of the narrowed section 33 is smaller than the equivalent diameter of the main channel section 35, along the direction from the first end 31 to the second end 32, at a first connection position D1 between the main channel section 35 and the narrowed section 33, the ventilation channel 3 transitions from the main channel section 35 to the narrowed section 33, and the equivalent diameter of the ventilation channel 3 abruptly decreases to form a narrowed portion. At a second connection position D2 between the main channel section 35 and the narrowed section 33, the ventilation channel 3 transitions from the narrowed section 33 to the main channel section 35, and the equivalent diameter of the ventilation channel 3 abruptly increases to form an expanded portion, so that during a liquid storage process, the capillary force direction A in the ventilation channel 3 is reversed at the expanded portion, to prevent the aerosol-forming medium from flowing out of the ventilation channel 3 from the second end 32 of the ventilation channel 3.

As shown in FIG. 11, in another implementation, the ventilation channel 3 may include only one main channel section 35 and one narrowed section 33, the main channel section 35 is at the first end 31, the narrowed section 33 is at the second end 32, and the equivalent diameter of the main channel section 35 is greater than the equivalent diameter of the narrowed section 33. The narrowed section 33 does not include the first narrowed portion 331 and the first expanded portion 332, and the equivalent diameter of the narrowed section 33 remains unchanged along a direction from the first end 31 to the second end 32 of the ventilation channel 3. Along the direction from the first end 31 to the second end 32, the ventilation channel 3 transitions from the main channel section 35 to the narrowed section 33. At a connection position between the main channel section 35 and the narrowed section 33 of the ventilation channel 3, the equivalent diameter of the ventilation channel 3 abruptly decreases to form a narrowed portion.

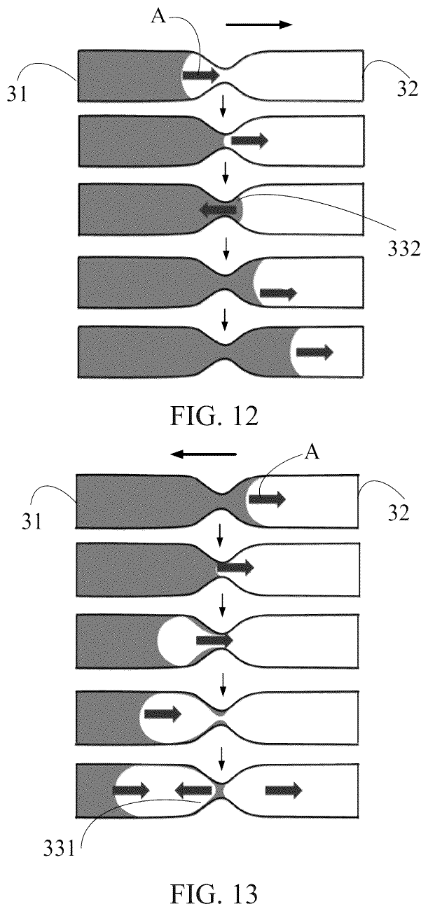

FIG. 12 is a schematic diagram of a structure of the ventilation channel shown in FIG. 4 during a liquid storage process, and FIG. 13 is a schematic diagram of a structure of the ventilation channel shown in FIG. 4 during a ventilation process.

In this embodiment, the side wall of the ventilation channel 3 has a lipophilic property, the first end 31 of the ventilation channel 3 is communicated with the liquid storage cavity 13, and the second end 32 is communicated with the atomization cavity 21. The narrowed section 33 is provided in the ventilation channel 3, and the capillary force direction A in the ventilation channel 3 changes at the first expanded portion 332, so that a problem that the aerosol-forming medium in the ventilation channel 3 flows out of the ventilation channel 3 from the second end 32 and then flows out of the atomizer 100 can be effectively prevented.

Specifically, as shown in FIG. 12, when the pressure in the liquid storage cavity 13 increases, the liquid storage process is performed in the ventilation channel 3, and the aerosol-forming medium in the ventilation channel 3 flows from the first end 31 to the second end 32, in other words, the aerosol-forming medium flows from the side where the liquid storage cavity 13 is located to the side where the atomization cavity 21 is located. In a process of the aerosol-forming medium flowing from the first end 31 to the second end 32, because the side wall of the ventilation channel 3 has a lipophilic property, at the main channel section 35 corresponding to the first end 31 of the ventilation channel 3, the capillary force direction A in the ventilation channel 3 points to the side where the atomization cavity 21 is located; and at the first expanded portion 332 of the narrowed section 33, the capillary force direction A in the ventilation channel 3 changes, and the capillary force direction A originally pointing to the side where the atomization cavity 21 is located changes to pointing to the side where the liquid storage cavity 13 is located. In other words, the original capillary force direction A points from the first end 31 to the second end 32, and when aerosol-forming medium passes through the first expanded portion 332, the capillary force direction A points from the second end 32 to the first end 31. The capillary force direction A at the first expanded portion 332 is opposite to the capillary force direction A at other positions. The capillary force pointing to the side where the liquid storage cavity 13 is located prevents the aerosol-forming medium in the ventilation channel 3 from continuing to flow toward the side where the atomization cavity 21 is located, to prevent a problem that the aerosol-forming medium in the ventilation channel 3 flows out of the ventilation channel 3 from the second end 32 of the ventilation channel 3 and then flows out of the atomizer 100.

As shown in FIG. 13, when the pressure in the liquid storage cavity 13 decreases, the ventilation process is performed in the ventilation channel 3 performs to realize the ventilation function. To be specific, during a process in which the air flows from the second end 32 to the first end 31 of the ventilation channel 3 to ventilate the liquid storage cavity 13, the aerosol-forming medium stored in the ventilation channel 3 flows from the second end 32 to the first end 31 and returns to the liquid storage cavity 13; because the side wall of the ventilation channel 3 has a lipophilic property, before the aerosol-forming medium flows through the first narrowed portion 331, the capillary force direction A points to the side where the atomization cavity 21 is located, in other words, the capillary force direction A points from the first end 31 to the second end 32. When the aerosol-forming medium passes through the first narrowed portion 331, the capillary force direction A changes, the air in the ventilation channel 3 flows to the first narrowed portion 331, and at the same time, the residual aerosol-forming medium in the ventilation channel 3 forms a thin liquid film at the first narrowed portion 331. An air-liquid mixing phenomenon occurs in the ventilation channel 3 when the air breaks through the thin liquid film subsequently, to complete the ventilation process.

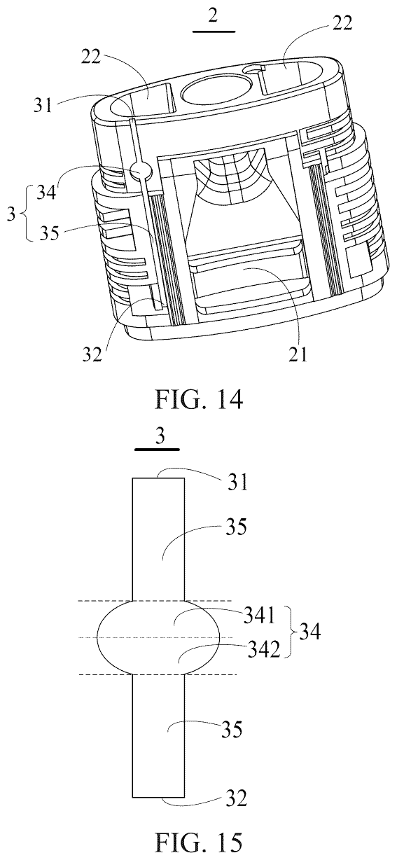

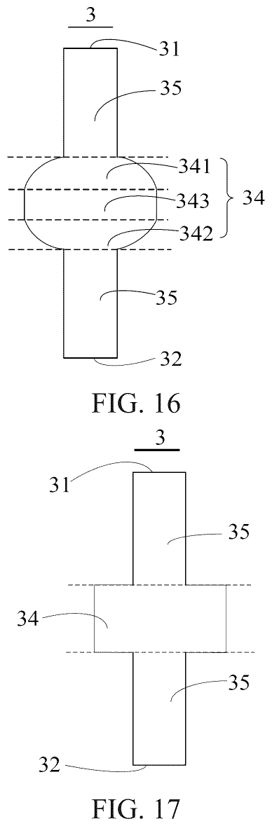

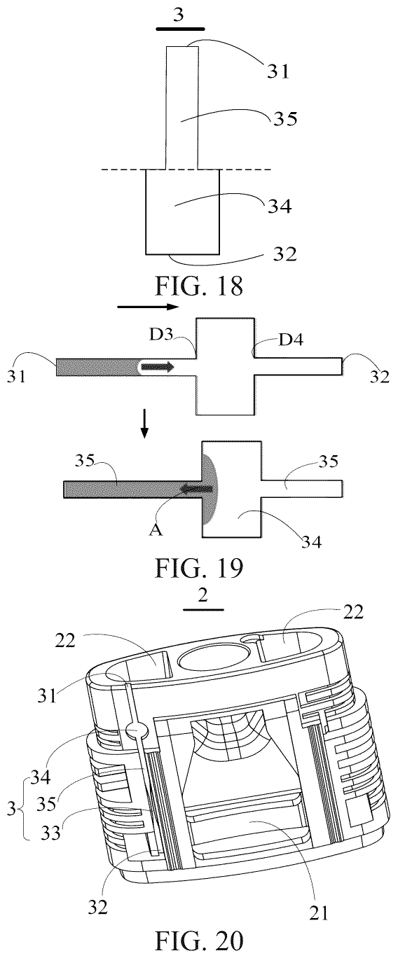

FIG. 14 is a schematic diagram of a structure of a second embodiment of a mounting base of the atomizer provided in FIG. 2; FIG. 15 is a schematic diagram of a structure of a first implementation of a ventilation channel of the mounting base provided in FIG. 14. FIG. 16 is a schematic diagram of a structure of a second implementation of a ventilation channel of the mounting base provided in FIG. 14. FIG. 17 is a schematic diagram of a structure of a third implementation of a ventilation channel of the mounting base provided in FIG. 14. FIG. 18 is a schematic diagram of a structure of a fourth implementation of a ventilation channel of the mounting base provided in FIG. 14. FIG. 19 is a schematic diagram of a structure of the ventilation channel shown in FIG. 17 during a liquid storage process.

Refer to FIG. 14. The ventilation channel 3 in this embodiment is formed in the same manner as the ventilation channel 3 in the first embodiment of the mounting base 2, both of which are formed by covering a micro groove in the mounting base 2 with a sealing member. Specifically, the sealing member and/or the mounting base 2 may be made of a lipophilic material, so that the formed ventilation channel 3 has a lipophilic property.

The mounting base 2 in this embodiment is different from the mounting base 2 in the first embodiment in that a structure of the ventilation channel 3 on the mounting base 2 in this embodiment is different from that in the first embodiment, and the remaining structures are the same as those in the first embodiment. Details are not described herein again.

Specifically, the ventilation channel 3 includes a plurality of main channel sections 35 and at least one expanded section 34. As shown in FIG. 14, the ventilation channel 3 includes two main channel sections 35 and one expanded section 34 communicated with each other. The expanded section 34 is located between the two main channel sections 35. The two main channel sections 35 are communicated with the liquid storage cavity 13 and the atomization cavity 21, respectively. In another implementation, the quantity of expanded sections 34 may be two or more. For example, the ventilation channel 3 may include three main channel sections 35 and two expanded sections 34. The two expanded sections 34 are communicated through one main channel section 35. The first end 31 and the second end 32 of the ventilation channel 3 are both main channel sections 35.

It may be understood that, when the ventilation channel 3 includes a plurality of expanded sections 34, during the liquid storage process in the ventilation channel 3, the plurality of expanded sections 34 can prevent the aerosol-forming medium from continuing to flow toward the second end 32. The resistance to the aerosol-forming medium is strong, and the effect is significant. In addition, the liquid storage volume in the ventilation channel 3 is large, so that the aerosol-forming medium in the ventilation channel 3 can be effectively prevented from flowing out from the second end 32.

In this embodiment, the narrowed portion and/or expanded portion may be directly formed at the expanded section 34, or may be jointly formed by the expanded section 34 and the main channel section 35.

Refer to FIG. 15. In one implementation, the narrowed portion and the expanded portion are directly formed at the expanded section 34. Specifically, the equivalent diameter of the expanded section 34 may first gradually increase and then gradually decrease along a direction from the first end 31 to the second end 32 of the ventilation channel 3. The expanded section 34 includes a second expanded portion 341 and a second narrowed portion 342. The equivalent diameter of the second expanded portion 341 gradually increases along the direction from the first end 31 to the second end 32 of the ventilation channel 3, and the equivalent diameter of the second narrowed portion 342 gradually decreases along the direction from the first end 31 to the second end 32 of the ventilation channel 3. In other words, the equivalent diameter at a position where the second expanded portion 341 is connected to the second narrowed portion 342 in the ventilation channel 3 is the largest, and the equivalent diameter of the expanded section 34 is greater than the equivalent diameter of the main channel section 35. The expanded section 34 may transition in any form such as a linear line transition form, a curve transition form, or a stepwise transition form along the direction from the first end 31 to the second end 32 of the ventilation channel 3.

As shown in FIG. 15, in one implementation, the expanded section 34 transitions in a curved form along the direction from the first end 31 to the second end 32 of the ventilation channel 3, and the side wall of the micro groove, which is provided on the outer side surface of the mounting base 2, at positions corresponding to the second narrowed portion 342 and the second expanded portion 341 is curved. Specifically, the side wall of the micro groove at the positions corresponding to the second narrowed portion 342 and the second expanded portion 341 are arcuate, and the side edges of the second narrowed portion 342 and the second expanded portion 341 on the longitudinal section of the expanded section 34 are arc lines. In another implementation, the expanded section 34 may alternatively use a linear line transition form or a stepwise transition form. The linear line transition form and the stepwise transition form are similar to the linear line transition form and the stepwise transition form of the narrowed section 33 in the first embodiment of the mounting base 2. Details are not described herein again.

Optionally, a second uniform connection portion 343 may also be connected between the second expanded portion 341 and the second narrowed portion 342. The equivalent diameter of the second uniform connection portion 343 remains unchanged along the direction from the first end 31 to the second end 32 of the ventilation channel 3, and the equivalent diameter of the second uniform connection portion 343 is the maximum equivalent diameter of the second expanded portion 341 and the second narrowed portion 342. The cross-sectional shape of the second uniform connection portion 343 may be any shape such as a rectangle, a circle, or a square.

As shown in FIG. 16, the expanded section 34 also includes the second uniform connection portion 343. The second uniform connection portion 343 is communicating the second expanded portion 341 and the second narrowed portion 342. The shapes of the second expanded portion 341 and the second narrowed portion 342 are the same as the shapes of the second expanded portion 341 and the second narrowed portion 342 in the expanded section 34 shown in

FIG. 14, and both transition in a curved form along the direction from the first end 31 to the second end 32 of the ventilation channel 3.

It may be understood that in another implementation, the expanded section 34 may alternatively include only the second expanded portion 341 but not the second narrowed portion 342. The second expanded portion 341 may transition in any form such as a linear line transition form, a curve transition form, or a stepwise transition form along the direction from the first end 31 to the second end 32.

In this implementation, the capillary force direction A in the ventilation channel 3 is reversed at the expanded section 34. Specifically, during the liquid storage process, the capillary force direction A is reversed at the second expanded portion 341 of the expanded section 34, to prevent the aerosol-forming medium in the ventilation channel 3 from continuing to flow toward the side where the atomization cavity 21 is located, so as to prevent leakage of the aerosol-forming medium caused by the aerosol-forming medium in the ventilation channel 3 flowing out of the ventilation channel 3 from the second end 32.

Refer to 17 and 18, in another implementation, the narrowed portion and/or expanded portion are/is jointly formed by the expanded section 34 and the main channel section 35.

Specifically, as shown in FIG. 17, in one implementation, the ventilation channel 3 includes two main channel sections 35 and one expanded section 34. The expanded section 34 is located between the two main channel sections 35. Sections at the first end 31 and the second end 32 are both the main channel sections 35. The equivalent diameter of the expanded section 34 may remain unchanged along the direction from the first end 31 to the second end 32 of the ventilation channel 3. To be specific, the expanded section 34 does not include the second expanded portion 341 and the second narrowed portion 342, and the equivalent diameter of the expanded section 34 is greater than the equivalent diameter of the main channel section 35. As shown in FIG. 17, the longitudinal section of the expanded section 34 is rectangular. To be specific, the shape of the side wall of the micro groove at the expanded section 34 on the mounting base 2 is a rectangular plane, and the equivalent diameter of the micro groove at the expanded section 34 is greater than the equivalent diameter at other positions.

Because the equivalent diameter of the expanded section 34 is greater than the equivalent diameter of the main channel section 35, along the direction from the first end 31 to the second end 32, at a third connection position D3 between the main channel section 35 and the expanded section 34, the ventilation channel 3 transitions from the main channel section 35 to the expanded section 34, and the equivalent diameter of the ventilation channel 3 abruptly increases to form an expanded portion. At a fourth connection position D4 between the main channel section 35 and the expanded section 34, the ventilation channel 3 transitions from the expanded section 34 to the main channel section 35, and the equivalent diameter of the ventilation channel 3 abruptly decreases to form a narrowed portion, so that during a liquid storage process, the capillary force direction A in the ventilation channel 3 is reversed at the expanded portion to prevent the aerosol-forming medium from flowing out of the ventilation channel 3 from the second end 32 of the ventilation channel 3.

As shown in FIG. 18, in another implementation, the ventilation channel 3 may include only one main channel section 35 and one expanded section 34, the main channel section 35 is at the first end 31, the expanded section 34 is at the second end 32, and the equivalent diameter of the expanded section 34 is greater than the equivalent diameter of the main channel section 35. The expanded section 34 does not include the second narrowed portion 342 and the second expanded portion 341, and the equivalent diameter of the expanded section 34 remains unchanged along a direction from the first end 31 to the second end 32 of the ventilation channel 3. Along the direction from the first end 31 to the second end 32, the ventilation channel 3 transitions from the main channel section 35 to the expanded section 34. At a connection position between the main channel section 35 and the expanded section 34 of the ventilation channel 3, the equivalent diameter of the ventilation channel 3 abruptly increases to form an expanded portion.

As shown in FIG. 19, the equivalent diameter of the expanded section 34 remains unchanged. When the pressure in the liquid storage cavity 13 increases, the liquid storage process is performed in the ventilation channel 3, and the aerosol-forming medium in the ventilation channel 3 flows from the first end 31 to the second end 32 of the ventilation channel 3. When the aerosol-forming medium flows in the main channel section 35, because the side wall of the ventilation channel 3 has a lipophilic property, the capillary force direction A in the ventilation channel 3 always points to the side where the atomization cavity 21 is located, in other words, the capillary force direction A points from the first end 31 to the second end 32, and the aerosol-forming medium is more likely to fill the ventilation channel 3. When the aerosol-forming medium flows from the main channel section 35 into the expanded section 34, the capillary force direction A is reversed, and the capillary force direction A points from the second end 32 to the first end 31, in other words, the capillary force direction A points the side where the liquid storage cavity 13 is located. The capillary force prevents the aerosol-forming medium from continuing to flow toward the side where the atomization cavity 21 is located, in other words, prevents the aerosol-forming medium from flowing toward the second end 32, so as to prevents the aerosol-forming medium in the ventilation channel 3 from flowing out from the second end 32 of the ventilation channel 3, thereby preventing leakage of the aerosol-forming medium in the ventilation channel 3. In addition, because the equivalent diameter of the expanded section 34 is larger, the liquid storage volume of the ventilation channel 3 is effectively increased, so that a problem that the aerosol-forming medium fills up the ventilation channel 3 and leaks from the second end 32 of the ventilation channel 3 is prevented, thereby effectively improving the performance of the atomizer 100.

FIG. 20 is a schematic diagram of a structure of a third embodiment of a mounting base of the atomizer provided in FIG. 2.

Refer to FIG. 20. The ventilation channel 3 in this embodiment is formed in the same manner as the ventilation channel 3 in the first embodiment of the mounting base 2, both of which are formed by covering a micro groove in the mounting base 2 with a sealing member. Specifically, the sealing member and/or the mounting base 2 may be made of a lipophilic material, so that the formed ventilation channel 3 has a lipophilic property.

The mounting base 2 in this embodiment is different from the mounting base 2 in the first embodiment in that a structure of the ventilation channel 3 on the mounting base 2 in this embodiment is different from that in the first embodiment, and the remaining structures are the same as those in the first embodiment of the mounting base 2. Details are not described herein again.

In this embodiment, the ventilation channel 3 includes both the narrowed section 33 and the expanded section 34. As shown in FIG. 20, the ventilation channel 3 includes three main channel sections 35, one narrowed section 33, and one expanded section 34 that are communicated with each other. The expanded section 34 is located on the side of the narrowed section 33 close to the first end 31. The expanded section 34 and the narrowed section 33 are communicated through the main channel section 35. In another implementation, the quantity of the main channel sections 35, the narrowed sections 33, and the expanded sections 34 may alternatively be configured to be any other quantity. For example, the ventilation channel 3 may include six main channel sections 35, two narrowed sections 33, and three expanded sections 34, the main channel sections 35 are at the first end 31 and the second end 32, and the remaining narrowed sections 33 and expanded sections 34 may be distributed at any positions.

Specifically, in this embodiment, a structure of the narrowed section 33 may be the structure of any implementation of the narrowed section 33 of the ventilation channel 3 in the first embodiment of the mounting base 2, and a structure of the expanded section 34 may also be the structure of any implementation of the expanded section 34 of the ventilation channel 3 in the second embodiment of the mounting base 2. It can be designed as needed, and this not limited in this application.

It may be understood that when the ventilation channel 3 includes both the narrowed section 33 and the expanded section 34, during the liquid storage process in the ventilation channel 3, both the narrowed sections 33 and the expanded section 34 can prevent the aerosol-forming medium from continuing to flow toward the second end 32. The resistance to the aerosol-forming medium is strong, and the effect is significant, so that the aerosol-forming medium in the ventilation channel 3 can be effectively prevented from flowing out from the second end 32, a prior art problem of leakage of the aerosol-forming medium in the ventilation channel 3 can be effectively resolved, thereby improving the performance of the atomizer 100 can be improved.

This application further provides another atomizer 100. In this embodiment, the atomizer 100 further includes a ventilation member. A ventilation channel 3 is provided in the ventilation member, and the ventilation member is disposed on a mounting base 2. The material of the ventilation member is a lipophilic material, so that the ventilation channel 3 has a lipophilic property.

Specifically, in one implementation, the ventilation member is a hollow tube. The hollow tube is of a linear structure and the hollow tube disposed on the mounting base 2. The hollow tube may be directly attached to the outer surface of the mounting base 2, so that the ports at the two ends of the hollow tube are respectively communicated with a liquid storage cavity 13 and the atmosphere, or the ports at the two ends of the hollow tube are respectively communicated with a liquid storage cavity 13 and an atomization cavity 21, so that the hollow tube is used as the ventilation channel 3.

Alternatively, a receiving groove may be provided on the side wall of the mounting base 2, and the hollow tube may be embedded in the receiving groove of the mounting base 2, so that the two ends of the hollow tube are directly communicated with a liquid storage cavity 13 and the atmosphere, respectively, or the two ends of the hollow tube are directly communicated with a liquid storage cavity 13 and an atomization cavity 21, respectively, so that the hollow tube is used as the ventilation channel 3. The hollow tube may be in any shape such as a hollow cylinder or a hollow prism.

In this embodiment, the hollow tube, namely, the ventilation channel 3, may include only a main channel section 35 and a narrowed section 33, or may include only a main channel section 35 and an expanded section 34, or the hollow tube may include a main channel section 35, a narrowed section 33, and an expanded section 34 at the same time, so that the ventilation channel 3 has a narrowed portion and/or an expanded portion. Specifically, a part of the wall of the hollow tube curves inward to form a narrowed section 33, in other words, the outer diameter and inner diameter of the hollow tube at the narrowed section 33 reduces, and/or a part of the wall of the hollow tube is expanded to form an expanded section 34, in other words, the outer diameter and inner diameter of the hollow tube at the expanded section 34 increases. Specific structures of the narrowed section 33 and the expanded section 34 of the hollow tube may be the same as the structures of the narrowed section 33 and the expanded section 34 in any of the foregoing implementations. Details are not described herein again. In another implementation, the ventilation member may alternatively be configured as a structural member of another shape, and the ventilation channel 3 is provided in the ventilation member.

It may be understood that the material of the ventilation member is a lipophilic material, so that the ventilation channel 3 has a lipophilic property, and a capillary force direction A in the ventilation channel 3 always points from a first end 31 to a second end 32. In this embodiment, the ventilation channel 3 is configured to be formed in the ventilation member, and the ventilation member is mounted on the mounting base 2. The ventilation channel 3 includes the narrowed section 33 and/or the expanded section 34, so that the ventilation channel 3 has a narrowed portion and/or an expanded portion. The capillary force direction A in the ventilation channel 3 is reversed at the narrowed section 33 and the expanded section 34. Specifically, the capillary force direction A is reversed at the narrowed portion or the expanded portion of the narrowed section 33 and/or expanded section 34 of the ventilation channel 3, to effectively prevent a prior art problem of leakage of the aerosol-forming medium caused by the aerosol-forming medium flowing out of the ventilation channel 3 from the second end 32 when the ventilation channel 3 is filled up with the aerosol-forming medium because the aerosol-forming medium in the ventilation channel 3 continuously flows from the first end 31 to the second end 32 due to the capillary force direction A in the ventilation channel 3 in the liquid storage cavity 13, which always points from the first end 31 to the second end 32.

In this embodiment, the ventilation member may be provided with only one ventilation channel 3 or may be provided with a plurality of ventilation channels 3. When the ventilation member is provided with only one ventilation channel 3, a prior art problem of leakage of the aerosol-forming medium caused by the asymmetric pressure in a plurality of ventilation channels 3 when the atomizer 100 includes the plurality of ventilation channels 3 can be resolved.

This application further provides another atomizer 100. In this embodiment, the atomizer 100 includes a liquid storage cavity 13 and a ventilation channel 3. The liquid storage cavity 13 is configured to store an aerosol-forming medium. A first end 31 of the ventilation channel 3 is communicated with the liquid storage cavity 13. The side wall of the ventilation channel 3 has a liquid-repellent property, for example, hydrophobicity (hydrophobicity) or oleophobicity (oleophobicity), to prevent the aerosol-forming medium from flowing out of the ventilation channel 3 from a second end 32 of the ventilation channel 3, thereby preventing a liquid leakage problem in the atomizer 100.

In other words, the atomizer 100 in this embodiment is different from the first and second embodiments of the atomizer 100 in that, in this embodiment, the aerosol-forming medium is an oily liquid, and the side wall of the ventilation channel 3 of the atomizer 100 has an oleophobic property.

Specifically, the ventilation channel 3 may be made of an oleophobic material. For example, the side wall of the ventilation channel 3 may be directly made of an oleophobic material, for example, PDMS or Teflon, or the side wall of the ventilation channel 3 may be formed by an oleophobic material prepared from a lipophilic material through treatment; or the side wall of the ventilation channel 3 may be coated with an oleophobic material coating so that the side wall of the ventilation channel 3 has an oleophobic property; or the side wall of the ventilation channel 3 may alternatively be configured to have an oleophobic micro-nano structure, so that the side wall of the ventilation channel 3 has an oleophobic property.

It may be understood that in this embodiment, the side wall of the ventilation channel 3 has an oleophobic property, so that during a flow process of the aerosol-forming medium in the ventilation channel 3, a capillary force direction A in the ventilation channel 3 always points to the side where the liquid storage cavity 13 is located. In other words, when a liquid storage process or a ventilation process is performed in the ventilation channel 3, due to the oleophobic property of the side wall of the ventilation channel 3, a capillary force direction A in the ventilation channel 3 always points from the second end 32 to the first end 31. When the liquid storage process is performed in the ventilation channel 3, that is, the pressure in the liquid storage cavity 13 increases, and the aerosol-forming medium flows from the first end 31 to the second end 32 of the ventilation channel 3, when the external temperature or pressure changes, the pressure difference between the inside and outside of the liquid storage cavity 13 changes, but the capillary force in the ventilation channel 3 overcomes the pressure difference between the inside and outside, to prevent the aerosol-forming medium from continuing to flow toward the second end 32 of the ventilation channel 3, so as to prevent liquid leakage caused by the aerosol-forming medium in the ventilation channel 3 flowing out of the ventilation channel 3 from the second end 32. When a ventilation process is performed in the ventilation channel 3, that is, the pressure in the liquid storage cavity 13 is low, and the external air and the aerosol-forming medium in the ventilation channel 3 flow from the second end 32 to the first end 31 of the ventilation channel 3, the capillary force direction A always points from the second end 32 to the first end 31. Under the joint action of the pressure difference between the inside and the outside and the capillary force, the along-the-way resistance of the flow process of the aerosol-forming medium in the ventilation channel 3 is overcome, so as to facilitate ventilation and achieve a smooth ventilation process and high ventilation efficiency, thereby reducing a risk of the atomizer 100 producing a burnt taste.

In this embodiment, the remaining structures of the atomizer 100 may be the same as the structures of the first embodiment or the second embodiment of the atomizer 100. Details are not described herein again. The ventilation channel 3 may be formed in any one of the manners in the first embodiment or the second embodiment of the atomizer 100. To be specific, the ventilation channel 3 may be directly formed in the mounting base 2. For example, the atomizer 100 includes a sealing member, and the outer side surface of the mounting base 2 is provided with a micro groove, and the sealing member covers the micro groove in the outer side surface of the mounting base 2 to form the ventilation channel 3. The material of the sealing member and/or the material of the mounting base 2 are/is an oleophobic material, so that the side wall of the ventilation channel 3 has an oleophobic property. Alternatively, the ventilation channel 3 may be configured to be formed in a separate structural member and the separate structural member may be mounted on the mounting base 2. For example, the atomizer 100 includes a ventilation member, and the ventilation member may be a hollow tube, so that the hollow tube is used as the ventilation channel 3. The material of the ventilation member is an oleophobic material so that the side wall of the ventilation channel 3 has an oleophobic property to prevent the aerosol-forming medium in the ventilation channel 3 from flowing out of the ventilation channel 3 from the second end 32.

The specific structure of the ventilation channel 3 may be the same as the structure of any implementation of the ventilation channel 3 in the first embodiment or the second embodiment of the atomizer 100. In other words, in this embodiment, the ventilation channel 3 may include a narrowed portion and/or an expanded portion. Because the side wall of the ventilation channel 3 in this embodiment has an oleophobic property, the capillary force direction A in the ventilation channel 3 points from the second end 32 to the first end 31 of the ventilation channel 3, and the capillary force direction A does not change at the expanded portion. Even if the capillary force direction A changes at the narrowed portion, the impact may be ignored because the capillary force direction A at other positions in the ventilation channel 3 always points to the first end 31, so that the aerosol-forming medium can be effectively prevented from flowing out of the ventilation channel 3 from the second end 32 of the ventilation channel 3.

It may be understood that in another implementation, the ventilation channel 3 may not include the narrowed portion and the expanded portion, and the ventilation channel 3 may only include a main channel section 35. In other words, the equivalent diameter of the ventilation channel 3 may remain unchanged along a direction from the first end 31 to the second end 32, or the equivalent diameter of the ventilation channel 3 may alternatively change in any form along the direction from the first end 31 to the second end 32, and the ventilation channel 3 may also be in any irregular shape, provided that the side wall of the ventilation channel 3 has an oleophobic property. In this embodiment, because the side wall of the ventilation channel 3 has an oleophobic property, even if the ventilation channel 3 does not include the narrowed portion and the expanded portion, the capillary force direction A in the ventilation channel 3 always points from the second end 32 to the first end 31 of the ventilation channel 3. The capillary force in the ventilation channel 3 prevents the aerosol-forming medium from continuing to flow toward the second end 32, so as to effectively prevent a liquid leakage problem caused by the aerosol-forming medium flowing out of the second end 32, thereby improving the performance of the atomizer 100.

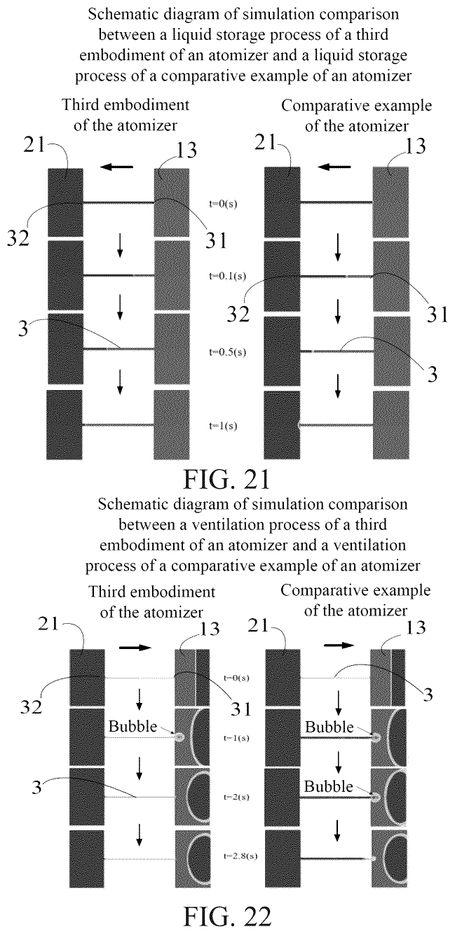

FIG. 21 is a schematic diagram of simulation comparison between a liquid storage process of a third embodiment of an atomizer and a liquid storage process of a comparative example of an atomizer; and FIG. 22 is a schematic diagram of simulation comparison between a ventilation process of a third embodiment of an atomizer and a ventilation process of a comparative example of an atomizer.

To verify the performance of a ventilation channel 3 of the third embodiment of the atomizer 100, that is, to verify the performance of a ventilation channel 3 having an oleophobic property, the inventor of this application respectively uses the atomizer 100 in the third embodiment and the comparison example of the atomizer to separately carry out experiments on the two different atomizers 100 under the same experimental conditions.

Specifically, the atomizer 100 in the third embodiment is only different from the comparative example of the atomizer in that the side wall of the ventilation channel 3 of the atomizer 100 in the third embodiment is made of an oleophobic material, while the side wall of the ventilation channel 3 of the comparative example of the atomizer is made of a lipophilic material. The rest of the structures of the atomizer 100 in the third embodiment and the comparative example of the atomizer are exactly the same. The same experiments are carried out on the two atomizers 100, and experimental results shown in FIG. 21 and FIG. 22 are obtained.

First, the comparative experiment is carried out on the liquid storage process in the ventilation channel 3 of the atomizer 100 in the third embodiment and the liquid storage process in the ventilation channel 3 of the comparative example of the atomizer under the same conditions. The experimental simulation results are shown in FIG. 21. The liquid storage process of the atomizer 100 (left side in FIG. 21) of the third embodiment and the liquid storage process of the comparative example of the atomizer (right side in FIG. 22) start at the same time at the second of t=0. In the comparative example of the atomizer on the right, the side wall of the ventilation channel 3 is of a lipophilic material, a capillary force direction A in the ventilation channel 3 points from a first end 31 to a second end 32, that is, a capillary force direction A points from a liquid storage cavity 13 to an atomization cavity 21/the external atmosphere, and the ventilation channel 3 actively absorbs liquid from the liquid storage cavity 13, so that an aerosol-forming medium flows faster in the ventilation channel 3 of the lipophilic material; while in the ventilation channel 3 of the atomizer 100 of the third embodiment on the left, because the side wall of the ventilation channel 3 is of an oleophobic material, a capillary force direction A in the ventilation channel 3 points from a second end 32 to a first end 31, and the capillary force prevents an aerosol-forming medium from flowing from the first end 31 to the second end 32, so that the aerosol-forming medium flows slowly in the ventilation channel 3 of the oleophobic material. At the second of t=1, the aerosol-forming medium in the atomizer 100 in the third embodiment on the left has not flowed to the second end 32 of the ventilation channel 3, while the aerosol-forming medium in the ventilation channel 3 of the atomizer in the comparative example on the right has already flowed out of the ventilation channel 3 from the second end 32 of the ventilation channel 3, resulting in a liquid leakage problem. After comparing and analyzing the experimental results, the applicant finds that when the ventilation channel 3 has an oleophobic property, a problem that the aerosol-forming medium from flows out of the ventilation channel 3 from the second end 32 of the ventilation channel 3 can be effectively prevented, so that a leakage problem of the atomizer 100 can be resolved.

The comparative experiment is carried out on the ventilation process in the ventilation channel 3 of the atomizer 100 in the third embodiment and the ventilation process in the ventilation channel 3 of the comparative example of the atomizer under the same conditions. The experimental simulation results are shown in FIG. 22. Under the same pressure, the ventilation process of the atomizer 100 (left side in FIG. 21) of the third embodiment and the ventilation process of the atomizer in the comparative example (right side in FIG. 22) start at the same time at the second of t=0. It can be seen from FIG. 22, in the ventilation channel 3 of the atomizer 100 of the third embodiment on the left, the ventilation process is completed at the second of t=2, while in the ventilation channel 3 of the comparative example of the atomizer on the right the ventilation process is completed at the second of t=2.8. In other words, the ventilation process in the ventilation channel 3 of the oleophobic material is completed faster than that of the ventilation channel 3 of the lipophilic material. After analyzing and comparing the experimental results, the inventor finds that the ventilation channel 3 of the atomizer 100 in the third embodiment has an oleophobic property, the capillary force direction A points to the side where the liquid storage cavity 13 is located, that is, from the second end 32 to the first end 31, and the capillary force and the pressure difference between the inside and the outside can work together to overcome the along-the-way resistance, to achieve a smooth ventilation process in the ventilation channel 3 and high ventilation efficiency, thereby reducing a risk of the atomizer 100 producing a burnt taste and improving the atomization performance.

After comparing and analyzing the experimental results of the liquid storage processes and the ventilation processes of the two atomizers 100 having the ventilation channels 3 having different characteristics, the inventor finally verifies that during the liquid storage process, the ventilation channel 3 having an oleophobic property can effectively prevent the aerosol-forming medium from flowing toward the second end 32 of the ventilation channel 3, to resolve a prior art problem of liquid leakage of the atomizer 100 caused by the aerosol-forming medium in the ventilation channel 3 continuously flowing toward the second end 32 and flowing out of the ventilation channel 3 from the second end 32 of the ventilation channel 3, thereby effectively improving the atomization performance of the atomizer 100. Moreover, during the ventilation process in the ventilation channel 3, the ventilation channel 3 having an oleophobic property may also make the capillary force and the force produced from the pressure difference between the inside and the outside act in the same direction, and the capillary force and the force produced from the pressure difference between the inside and the outside jointly overcome the along-the-way resistance when the aerosol-forming medium flows from the second end 32 to the first end 31, to facilitate ventilation and achieve a smooth ventilation process and high ventilation efficiency, so that a prior art problem of ventilation difficulty in the ventilation channel is effectively resolved, thereby reducing a risk of the atomizer 100 producing a burnt taste.

Different from the prior art, this application discloses an atomizer 100 and an electronic atomization apparatus 300. The atomizer 100 includes a liquid storage cavity 13 and a ventilation channel 3. The liquid storage cavity 13 is configured to store an aerosol-forming medium. A first end 31 of the ventilation channel 3 is communicated with the liquid storage cavity 13. The ventilation channel 3 includes a narrowed portion and/or an expanded portion, to prevent the aerosol-forming medium from flowing out of the ventilation channel 3 from a second end 32 of the ventilation channel 3. Based on the foregoing configuration, a structure of the narrowed portion and/or the expanded portion of the ventilation channel 3 changes the capillary force direction A in the ventilation channel 3, effectively preventing the aerosol-forming medium in the ventilation channel 3 from flowing out of the ventilation channel 3 from the second end 32, thereby resolving a prior art problem that the aerosol-forming medium in the ventilation channel is prone to leakage and improving the performance of the atomizer 100.

The foregoing descriptions are merely embodiments of this application, and the patent scope of this application is not limited thereto. All equivalent structure or process changes made based on the content of this specification and accompanying drawings in this application or by directly or indirectly applying this application in other related technical fields shall fall within the patent protection scope of this application.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. An atomizer, comprising:
a liquid storage cavity configured to store an aerosol-forming medium; and
a ventilation channel, a first end of which is communicated with the liquid storage cavity, the ventilation channel comprising a narrowed portion and/or an expanded portion configured to prevent the aerosol-forming medium from flowing out of the ventilation channel from a second end of the ventilation channel.

2. The atomizer of claim 1, wherein the ventilation channel comprises a main channel section and at least one narrowed section that are communicated with each other, the narrowed section comprising the narrowed portion and/or the expanded portion, or the narrowed section and the main channel section jointly forming the narrowed portion and/or the expanded portion, and/or
wherein the ventilation channel comprises a main channel section and at least one expanded section that are communicated with each other, the expanded section comprising the narrowed portion and/or the expanded portion, or the narrowed section and the main channel section jointly forming the narrowed portion and/or the expanded portion.

3. The atomizer of claim 2, wherein the ventilation channel comprises the narrowed section, and an equivalent diameter of the narrowed section decreases along a direction from the first end of the ventilation channel to the second end of the ventilation channel so as to form a first narrowed portion, and then increases to form a first expanded portion, or an equivalent diameter of the narrowed section remains unchanged along a direction from the first end of the ventilation channel to the second end of the ventilation channel, and the equivalent diameter of the narrowed section is smaller than an equivalent diameter of the main channel section, or
wherein the ventilation channel comprises the expanded section, and an equivalent diameter of the expanded section increases along a direction from the first end of the ventilation channel to the second end of the ventilation channel so as to form a second expanded portion, and then decreases so as to form a second narrowed portion, or an equivalent diameter of the expanded section remains unchanged along a direction from the first end of the ventilation channel to the second end of the ventilation channel, and an equivalent diameter of the expanded section is greater than an equivalent diameter of the main channel section.

4. The atomizer of claim 3, wherein the ventilation channel comprises the narrowed section, the narrowed section comprising a first uniform connection portion communicating the first narrowed portion and the first expanded portion, and an equivalent diameter of the first uniform connection portion remains unchanged along the direction from the first end of the ventilation channel to the second end of the ventilation channel, or
wherein the ventilation channel comprises the expanded section, the expanded section comprising a second uniform connection portion communicating the second narrowed portion and the second expanded portion, and an equivalent diameter of the second uniform connection portion remains unchanged along a direction from the first end of the ventilation channel to the second end of the ventilation channel.

5. The atomizer of claim 2, wherein the ventilation channel comprises the main channel section and the at least one narrowed section that are communicated with each other, and
wherein a maximum equivalent diameter of the narrowed section is 0.2 mm to 1 mm.

6. The atomizer of claim 2, wherein the ventilation channel comprises the main channel section and the at least one narrowed section that are communicated with each other,
wherein a minimum equivalent diameter of the narrowed section is smaller than a maximum equivalent diameter, and
wherein a ratio of the maximum equivalent diameter to the minimum equivalent diameter of the narrowed section is M, wherein 1<M<4.

7. The atomizer of claim 1, wherein the narrowed portion or the expanded portion transitions in a curved form along a direction from the first end of the ventilation channel to the second end of the ventilation channel, or
wherein the narrowed portion or the expanded portion transitions in a linear form along a direction from the first end of the ventilation channel to the second end of the ventilation channel, or
wherein the narrowed portion or the expanded portion transitions in a stepwise form along a direction from the first end of the ventilation channel to the second end of the ventilation channel.

8. The atomizer of claim 1, wherein a side wall of the ventilation channel is lyophilic.

9. The atomizer of claim 8, wherein a material of the side wall of the ventilation channel comprises a lipophilic material, or
wherein a side wall of the ventilation channel comprises a lipophilic material coating, or
wherein a side wall of the ventilation channel comprises a lipophilic micro-nano structure.

10. The atomizer of claim 1, further comprising:
a housing provided with an air outlet tube and an accommodating cavity; and
a mounting base disposed in the accommodating cavity, the mounting base and the housing jointly forming the liquid storage cavity, the mounting base being internally provided with an atomization cavity, the atomization cavity being communicated with the air outlet tube and external atmosphere,
wherein the ventilation channel is provided in the mounting base, and
wherein a second end of the ventilation channel is communicated with the atomization cavity or the external atmosphere.

11. The atomizer of claim 10, wherein the ventilation channel comprises a linear structure, and
wherein a first end of the ventilation channel is directly communicated with the liquid storage cavity, and the second end of the ventilation channel is communicated with the atomization cavity.

12. The atomizer of claim 10, further comprising:
a sealing member, an outer side surface of the mounting base being provided with a micro groove, the sealing member covering the micro groove to form the ventilation channel; or
a ventilation member, the ventilation channel being provided in the ventilation member, the ventilation member being disposed on the mounting base.

13. The atomizer of claim 12, wherein the atomizer comprises the ventilation member, and the ventilation member comprises a hollow tube so as to form the ventilation channel,
wherein a part of a wall of the hollow tube curves inward to form a narrowed section, the narrowed section comprising a first narrowed portion and a first expanded portion, and/or
wherein a part of a wall of the hollow tube is expanded outward to form an expanded section, the expanded section comprising a second narrowed portion and a second expanded portion.

14. The atomizer of claim 12, wherein a material of the sealing member and/or a material of the mounting base comprises/comprise a lipophilic material, or
wherein a material of the ventilation member comprises a lipophilic material.

15. The atomizer of claim 10, further comprising:
an atomization core disposed in the mounting base and fluidly communicated with the liquid storage cavity, the atomization core comprising a porous liquid-guiding substrate and a heating element.

16. The atomizer of claim 1, wherein the atomizer comprises only one ventilation channel.

17. An electronic atomization apparatus, comprising:
the atomizer of claim 1; and
a power supply assembly electrically connected to the atomizer, the power supply assembly being configured to provide energy to the atomizer.
